(19) United States
(12) Patent Application Publication
(10) Pub. No.: US 2010/0160339 A1
(43) Pub. Date: Jun. 24, 2010

(54) TREATING PANCREATITIS

(75) Inventors: Mark A. McNiven, Rochester, MN (US); Vijay P. Singh, Pittsburgh, PA (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/600,886

(22) PCT Filed: May 13, 2008
(86) PCT No.: PCT/US08/63543

§ 371 Date: Nov. 19, 2009

Related U.S. Application Data
(60) Provisional application No. 60/939,240, filed on May 21, 2007.

(51) Int. Cl. A61K 31/496; A61P 1/18

(52) U.S. Cl. 514/252.18

(57) ABSTRACT

This document provides methods and materials related to treating pancreatitis. For example, methods and materials relating to the use of a tyrosine kinase inhibitor to pancreatitis are provided.

### STATEMENT AS TO FEDERALLY SPONSORED RESEARCH

This invention was made with government support under 5T32DK07198-31 awarded by the National Institutes of Health. The government has certain rights in the invention.

### BACKGROUND

1. Technical Field

This document relates to methods and materials involved in treating pancreatitis. For example, this document relates to methods and materials involved in using a tyrosine kinase inhibitor (e.g., a Src inhibitor such as PP2 or SU6656) to treat pancreatitis.

2. Background Information

The pancreas is a large gland located near the stomach and the duodenum. The pancreas releases digestive enzymes into the small intestine through the pancreatic duct. The digestive enzymes can digest fats, polypeptides, and carbohydrates present in food. Under normal conditions, the digestive enzymes are inactive until they reach the small intestine. In some cases, however, the digestive enzymes can become active inside the pancreas and can start digesting the pancreas itself.

### SUMMARY

This document provides methods and materials related to treating pancreatitis. For example, this document relates to methods and materials involved in using a tyrosine kinase inhibitor (e.g., a Src inhibitor such as PP2 or SU6656) to treat pancreatitis. The term “pancreatitis” as used herein refers to any acute or chronic inflammation of the pancreas. Pancreatitis can be asymptomatic or symptomatic and can be due to autodigestion of pancreatic tissue by digestive enzymes from the pancreas. Pancreatitis can be caused by alcoholism or biliary tract disease and can be associated with hyperlipaemia, hyperparathyroidism, abdominal trauma (e.g., an accidental or operative injury), vasculitis, or uraemia. Both acute and chronic pancreatitis can cause serious complications. In severe cases, bleeding, tissue damage, and infection can occur, and enzymes and toxins can enter the blood and injure other organs such as the heart, lungs, and kidneys. The methods and materials provided here can allow clinicians to treat a mammal having pancreatitis or suspected of developing pancreatitis, thereby providing the mammal with a healthier quality of life.

In one aspect, this document features a method for treating a mammal having pancreatitis. The method comprises, or consists essentially of, administering a tyrosine kinase inhibitor having the ability to inhibit phosphorylation of cortactin to the mammal under conditions wherein the severity of a symptom of the pancreatitis is reduced. The mammal can be a human. The pancreatitis can be acute pancreatitis. The pancreatitis can be chronic pancreatitis. The tyrosine kinase inhibitor can be selected from the group consisting of SKI-606, BMS354825, AZD0530, AP23464, CGP76030, AMN107, PP1, PP2, SU6656, and Imatinib mesylate. The method can comprise administering a composition comprising two or more tyrosine kinase inhibitors having the ability to inhibit phosphorylation of cortactin. The severity of the symptom can be reduced by at least 25 percent. The severity of the symptom can be reduced by at least 50 percent. The severity of the symptom can be reduced by at least 75 percent. The method can comprise identifying the mammal as having the pancreatitis before the administering step. The method can comprise monitoring the mammal for the reduction in the severity of the symptom after the administering step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 provides results demonstrating that suprastimulation induces cortactin phosphorylation and its basal relocation and contains immunofluorescence images of cortactin and actin, and western blots of immunoprecipitated cortactin showing its tyrosine phosphorylation. Resting acini have apical actin (a) and some apical cortactin (b). Suprastimulation with 10 nM CCK resulted in blebbing and basal relocation of actin (a″) and cortactin (b″) unlike physiologic stimulation (0.1 nM CCK) which did not affect the apical actin (a′) and enriched the apical cortactin (b′) without causing blebbing. (c) Acini were stimulated with logarithmically increasing doses of CCK for 2 minutes and lysed, and immunoprecipitated cortactin was blotted with a phosphotyrosine antibody (4G10). Significantly increased cortactin phosphorylation occurred at doses of 10 nM CCK or higher. (d) A time course of cortactin tyrosine phosphorylation after stimulation with 10 nM CCK is provided.

FIG. 2 provides results demonstrating that Src mediated cortactin phosphorylation induced by suprastimulation causes the Src-cortactin complex to dissociate resulting in the basal relocation of cortactin. FIG. 2 contains immunofluorescence and co-immunoprecipitation of src and cortactin after CCK treatment. Src (using pan Src antibody) and cortactin existed as a complex localizing to the apical region (arrow a, a′) under control conditions (also f “bas” and g time “0”). With suprastimulation (10 nM CCK, which induced cortactin phosphorylation) cortactin moved basally within five minutes (b′), and this is pronounced at 30 minutes (c′ arrow) when it localized to the base of the blebs. Src in contrast did not localize to the basal membrane under these conditions (b, c). PP2 prevented this basal redistribution of cortactin (d′), and it remained apical with Src (d). 0.1 nM CCK (e, e′) (which did not induce cortactin phosphorylation) in contrast enhanced the apical localization of cortactin. (f) A dose response of the dissociation of the Src-cortactin complex 2 minutes after different concentration of CCK as detected after immunoprecipitating Src (src) and blotting for cortactin (Cort) is provided. There was complete dissociation of the complex at 10 nM CCK. This was prevented by PP2. The graph provides the dose response of dissociation of the complex in relation to Src activation (as fold control) and cortactin phosphorylation taking unstimulated controls as 100%. (g) A time course of src activation and dissociation of the src-cortactin complex as detected by immunoprecipitating Src and blotting for active src (PY416) and cortactin (Cort) after stimulation with 10 nM CCK is provided. The graph shows the dissociation of the complex in relation to cortactin phosphorylation taking unstimulated controls as 100% (g).

FIG. 3. The Src family member Yes interacts with cortactin. (a) Acinar cell lysates and immunoprecipitates from control cells using the pan Src antibody blotted for different src family members. Yes was the only src family member pulled down by the pan src antibody. The immunoprecipitates were negative for fyn, S-Src, and Lyn, even though these were present in the whole cell lysates. (b) Acinar cell lysates and immunoprecipitates from control cells using the cortactin antibody blotted for cortactin (top lane) and different Src family members. Yes is the only src family member that binds cortactin. The immunoprecipitates were negative for fyn, S-Src, and Lyn. (c) Dose response of src activation in acinar cells. Whole cell lysates (top panel) were blotted for active src (PY416) and reprobed for Yes. Yes overlapped the active src bands in the whole cell lysates. Immunoprecipitates for Yes (bottom panel) blotted for active src (PY416) and Yes. The activation of src is identical to that of Yes in whole cell lysates. (d-g) Immunofluorescence of cortactin and Yes in acini; effect of CCK and Src inhibition with PP2. Yes immunofluorescence like that of total src remained apical at physiologic (0.1 nM) and supraphysiologic (10 nM CCK) concentrations (d′, e′, f). Cortactin in contrast moved basally with supraphysiologic CCK (f; compared to d and e). PP2 prevented this basal relocation of cortactin induced by 10 nM CCK (g).

FIG. 4. Src inhibition prevents suprastimulation induced basal localization of cortactin and actin, resulting in reduced number of blebs. Phase images of acini suprastimulated with CCK (10 nM) forming blebs (a-a″) are provided. Corresponding changes in cortactin (b) and actin (b′), respectively, are provided. The src inhibitors PP2 and SU665 prevented bleb formation (c-c″ and e-e″, respectively), and the corresponding basolateral localization of cortactin and actin (d, d′ and f, f′, respectively). (g) The effects of PP2 and SU6656 in reducing the number of blebs was quantified. A total of 299 cells were counted for 10 nM CCK, 180 for 10 nM CCK+PP2, and 160 for 10 nM CCK+SU6656. (h, i) PP2 and SU6656 prevented 10 nM CCK induced phosphorylation of cortactin and number of blebs induced by CCK (i).

FIG. 5. Cortactin refractory to Src induced tyrosine phosphorylation (M3 cortactin) reduces suprastimulation-induced blebbing. Immunofluorescence photographs of cortactin (a) and actin (a′) and corresponding brightfield images with merged immunofluorescence (a″) of an untranfected acinus (right) and an acinus transfected with M3 cortactin (left acinus) after stimulation with 10 nM CCK for 30 minutes are provided. While the untranfected acinus reorganizes its actin to the basal surface (white arrow) and forms large blebs. The M3 transfected acinus retained its apical actin (black border arrowhead) and did not form blebs. (b) A graph showing reduction in 10 nM CCK induced blebs by M3 cortactin compared to untransfected acini and WT cortactin transfected acini is provided. A time course of phosphorylation of over-expressed wild type cortactin after 10 nM CCK (c, upper panel) is provided. While over-expressed wild type cortactin was phosphorylated like endogenous cortactin, M3 cortactin was not (c, lower panel). Immunoprecipitates of cortactin prepared from acini stimulated for 1 minute with 10 nM CCK was blotted for Yes (d). Yes dissociated from wild type cortactin, while it remains bound to M3 cortactin at this time.

FIG. 6. Src induced cortactin phosphorylation occurs during pancreatitis and causes the basal localization of actin and cortactin. (a) Lysates prepared from pancreata of animals after different times of CCK administration (20 μg/kg) were blotted for active src (PY416) and Yes. (b) Immunoprecipitates of cortactin prepared from the same pancreata as in (a) were blotted for phosphotyrosine (4G10) and cortactin (Cort). (c) A graphical representation of time course of Src activation in pancreatic lysates is provided. (d) Immunoprecipitates of cortactin prepared from pancreata of untreated animals (con), 20 μg/kg CCK 30 minutes before (Cer 30), or CCK preceded by 3 mg/kg PP2 by 30 minutes (cer+PP2) were blotted for phosphotyrosine (WB 4G10) or cortactin (WB cort). Immunofluorescence of sections from the pancreas were stained for actin (e, f, g) and cortactin (e′, f, g′) from control animals (control) or animals administered 20 μg/kg CCK 30 minutes before (Cer, 30 min) or pretreated with PP2 (3 mg/kg) 30 minutes prior to CCK administration (Cer30+PP2). PP2 restored apical co-localization of actin and cortactin.

FIG. 7. Src inhibition reduces the severity of suprastimulation-induced pancreatitis. Serum amylase (a), pancreatic water content (b), and pancreatic histologies (c, d, e) were determined for untreated animals (con), those given a single IP injection of CCK (20 μg/kg) and sacrificed 6 hours later (CER), those administered 0.1 mL DMSO intra-peritoneally 30 minutes before CCK and sacrificed 6 hours after the CCK injection (CER+Veh.), or those given 3 mg/Kg PP2 dissolved in 0.1 mL DMSO, 30 minutes before the CCK injection and sacrificed 6 hours after the CCK injection (CER+PP2).

### DETAILED DESCRIPTION

This document provides methods and materials related to treating pancreatitis in mammals. For example, this document provides methods and materials related to the use of a tyrosine kinase inhibitor to treat pancreatitis in a mammal. The methods and materials provided herein can be used to treat pancreatitis in any type of mammal including, without limitation, mice, rats, dogs, cats, horses, cows, pigs, monkeys, and humans. Any type of pancreatitis, such as acute or chronic pancreatitis, can be treated. In some cases, a symptomatic, acute pancreatitis can be treated.

In general, pancreatitis can be treated by administering a tyrosine kinase inhibitor to a mammal having pancreatitis. It will be appreciated that a single tyrosine kinase inhibitor or a combination of tyrosine kinase inhibitors (e.g., two, three, four, five, or more tyrosine kinase inhibitors) can be used to treat pancreatitis upon administration. For example, a mammal having pancreatitis can be treated by administering a composition containing scr and Yes inhibitors to a mammal.

Any tyrosine kinase inhibitor, such as scr inhibitor, Abl inhibitors and Yes inhibitors, can be used to treat pancreatitis. Examples of tyrosine kinase inhibitors that can be used to treat pancreatitis include, without limitation, SKI-606 (Wyeth-Ayerst), BMS354825 (Bristol-Myers), AZD0530 (Astra Zeneca), AP23464 (Ariad), CGP76030 (Pfizer), AMN107 (Novartis), PP1 (4-Amino-5-(4-methylphenyl)-7-(t-butyl)pyrazolo[3,4-d]-pyrimidine), PP2 (Calbiochem), SU6656 (Calbiochem), and Imatinib mesylate (also called Gleevec® or STI571; Novartis).

Any appropriate method can be used to administer a tyrosine kinase inhibitor to a mammal. For example, a tyrosine kinase inhibitor can be administered orally or via injection (e.g., subcutaneous injection, intramuscular injection, intravenous injection, or intrathecal injection). In some cases, a combination of tyrosine kinase inhibitors can be administered by different routes. For example, one tyrosine kinase inhibitor can be administered orally and a second tyrosine kinase inhibitor can be administered via injection.

Before administering a tyrosine kinase inhibitor to a mammal, the mammal can be assessed to determine whether or not the mammal has pancreatitis. Any appropriate method can be used to determine whether or not a mammal has pancreatitis. For example, a mammal (e.g., human) can be identified as having pancreatitis using standard diagnostic techniques. In some cases, a diagnostic imaging techniques can be used to determine whether or not a mammal has pancreatitis.

After identifying a mammal as having pancreatitis, the mammal can be administered a tyrosine kinase inhibitor. A tyrosine kinase inhibitor can be administered to a mammal in any amount, at any frequency, and for any duration effective to achieve a desired outcome (e.g., to reduce a symptom of pancreatitis). In some cases, a tyrosine kinase inhibitor can be administered to a mammal having pancreatitis to reduce a symptom of pancreatitis 5, 10, 25, 50, 75, 80, 85, 90, 95, or 100 percent. Any method can be used to determine whether or not the severity of a symptom of pancreatitis is reduced. For example, the severity of a symptom of pancreatitis can be assessed by imaging tissue at different time points and determining the amount of inflammation present. The amounts of inflammation determined within tissue at different times can be compared to determine the level of reduction in inflammation.

An effective amount of a tyrosine kinase inhibitor can be any amount that reduces the severity of a symptom of pancreatitis without producing significant toxicity to the mammal. For example, an effective amount of a tyrosine kinase inhibitor can be from about 0.05 mg/kg to about 100 mg/kg (e.g., from about 0.1 mg/kg to about 50 mg/kg, from about 0.2 mg/kg to about 25 mg/kg, or from about 0.5 mg/kg to about 10 mg/kg). Typically, an effective amount of a tyrosine kinase inhibitor such as PP1 or PP2, both of which are nearly equipotent and belong to the pyrazolo[3,4-d]pyrimidine class of agents, can be from about 0.2 mg/kg to about 10 mg/kg (Khadaroo et al., Surgery, 136:483-488 (2004) and Dickerson and Sharp, Neuropsychopharm., 31(7):1420-30 (2006)). If a particular mammal fails to respond to a particular amount, then the amount of tyrosine kinase inhibitor can be increased by, for example, two fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the pancreatitis may require an increase or decrease in the actual effective amount administered.

The frequency of administration can be any frequency that reduces the severity of a symptom of pancreatitis without producing significant toxicity to the mammal. For example, the frequency of administration can be from about once a week to about three times a day, or from about twice a month to about six times a day, or from about twice a week to about once a day. The frequency of administration can remain constant or can be variable during the duration of treatment. A course of treatment with a tyrosine kinase inhibitor can include rest periods. For example, a tyrosine kinase inhibitor can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the pancreatitis may require an increase or decrease in administration frequency.

An effective duration for administering a tyrosine kinase inhibitor can be any duration that reduces the severity of a symptom of pancreatitis without producing significant toxicity to the mammal. Thus, the effective duration can vary from several days to several weeks, months, or years. In general, the effective duration for the treatment of pancreatitis can range in duration from several weeks to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the pancreatitis.

A composition containing a tyrosine kinase inhibitor can be in any appropriate form. For example, a composition provided herein can be in the form of a solution or powder with or without a diluent to make an injectable suspension. A composition also can contain additional ingredients including, without limitation, pharmaceutically acceptable vehicles. A pharmaceutically acceptable vehicle can be, for example, saline, water, lactic acid, and mannitol.

After administering a composition provided herein to a mammal, the mammal can be monitored to determine whether or not the pancreatitis was treated. For example, a mammal can be assessed after treatment to determine whether or not the severity of a symptom of pancreatitis was reduced.

The invention will be further described in the following examples, which do not limit the scope of the invention described in the claims.

### EXAMPLES

### Example 1

### Treating Pancreatitis with Tyrosine Kinase Inhibitors

### Methods and Materials

Male 100-120 gm Sprauge Dawley (Harlan Laboratories, Indianapolis, Ind.) mice that were housed and fed under standard conditions were used. Caerulein, the CCK analog, was obtained from Research Plus (Bayonne, N.J.). Antibodies to phosphotyrosine (4G10) and cortactin (4F11) antibodies were obtained from Upstate (Charlottesville, Va.). Pan Src antibody (SC-18), Fyn (SC-16) and c-Src antibody H-12 were obtained from Santa Cruz (Santa Cruz, Calif.), and active Src (PY416), Yes and Lyn antibodies were obtained from Transduction Labs (Lexington, Ky.). Polyclonal antibody to cortactin (AB3) was produced as described elsewhere (Cao et al., Mol. Cell Biol., 23(6):2162-2170 (2003)). Secondary antibodies for immunofluorescence (Alexa 488 and Alexa 594) and Prolong gold were obtained from Invitrogen Corporation (Carlsbad, Calif.). Secondary antibodies for western blotting (goat anti-rabbit and goat anti-mouse HRP) were obtained from Affinity Bioreagents (Golden, Colo.). Collagenase type IV was obtained from Worthington Biochemical Corporation (Lakewood, N.J.). PP2 and SU6656 were obtained from Calbiochem (San Diego, Calif.) and were dissolved as a 1000× stock in DMSO. All other reagents were purchased from Sigma.

### Preparation and Use of Acini

Acini were harvested in a buffer containing 20 mM HEPES (pH 7.4), 120 mM NaCl, 5 mM KCl, 1 mM MgCl₂, 1 mM CaCl₂, 10 mM glucose, 10 mM sodium pyruvate, 0.1% bovine serum albumin, and 0.01% soybean trypsin inhibitor with 100 units/mL collagenase, as described elsewhere (Torgerson and McNiven, J. Cell Sci., 111 (Pt. 19):2911-22 (1998)) were filtered through a 70 μm mesh and kept at 37° C. for two hours before use during which time these were pre-incubated with 20 μM of inhibitors (PP2 or SU6656), if necessary. Viability prior to use was >95% by trypan blue exclusion. The harvested acini were used as described elsewhere (Torgerson and McNiven, J. Cell Sci., 111 (Pt. 19):2911-22 (1998)).

For culture experiments involving transfection, these cells were cultured for 16 hours in RPMI 1640 with 10% FCS, 20 mM Nicotinamide, and antibiotics (pen/strept). Adenovirus was prepared by the University of Iowa, Gene Transfer Vector Core using the RAPAd system (U.S. Pat. No. 6,830,920). Viruses encoding for wild-type cortactin (WT-cortactin) and a tyrosine to phenylalanine mutant at positions 384, 429, and 445 (M3 cortactin) were incubated with cells at 10⁷ pfu/mL. To detect the expression of either WT-cortactin or M3 cortactin polypeptide, cells were processed for immunofluorescence or western blotting 16 hours later using the 4F11 or AB3 antibody, respectively.

### Immunofluorescence Staining

Acini were plated on plain glass coverslips as described elsewhere (Torgerson and McNiven, J. Cell Sci., 111 (Pt. 19):2911-22 (1998)), fixed with 2% paraformaldehyde, and permeabilized with 0.6% Triton X-100. The acini were then blocked with 5% normal goat serum followed by incubation with the primary antibodies (4F11 diluted 1/1000, SC-18 diluted 1/300, or anti-Yes diluted 1/1000) for one hour at room temperature followed by washing and incubation with secondary antibodies ( 1/500) with or without rhodamine phalloidin (100 nM) for 30 minutes. The acini were then washed and mounted using Prolong gold. Acini with 6-12 cells were randomly chosen for imaging. The number of blebs was counted along with the number of cells, and expressed as bleb number per 100 cells. The final number expressed as mean±SEM was from at least three different experiments. Imaging was performed using an LSM510 microscope from Zeiss, and TIF images were processed in Adobe photoshop 6.0 to separate channels and process.

### Immunoprecipitation and Western Blotting

Acini or tissue were homogenized in a buffer containing 50 mM Tris (pH 7.2), 150 mM NaCl, 0.5 mM EDTA, 1 mM EGTA, 2 mM DTT, 1 mM sodium orthovanadate, 25 mM NaF, and 1% NP-40, using a protease inhibitor cocktail (Complete; Cat. # 1873580; Roche) to prevent proteolysis. The homogenized samples were boiled in 1× Lamelli buffer for western blotting as described elsewhere (Cao et al., Mol. Cell Biol., 23(6):2162-2170 (2003)) or immunoprecipitated for two hours at 4° C. using 5 μg/mL of primary antibody followed by 4 mg protein A beads for one hour in the same buffer. The beads were washed three times and boiled in 1× Lamelli buffer. Supernatants were used for Western blotting. After Western blotting using appropriate protocols and dilutions of antibodies ( 1/100000 for AB3, 1/1000 for 4G10, 1/500 for SC-18, 1/1000 for PY416, 1/5000 for Yes, 1/200 for H-12 (c-Src), 1/200 for fyn, and 1/200 for Lyn) as recommended by the manufacturer, band intensity was quantified using Adobe Photoshop 6.0, and backgrounds were subtracted for the appropriate lanes. The loading control used was the intensity of the band of the polypeptide whose phosphorylation was being studied or the one which was being primarily immunoprecipitated. Quantification was performed from two or more experiments.

### Blood and Tissue Preparation

Animals (6 animals in each group) were given a single intraperitoneal injection of saline (control) or caerulein (20 μg/kg) in saline as described elsewhere (Bhagat et al., Gastroenterology, 122(1): 156-165 (2002)). After 6 hours, blood and tissue samples were harvested and frozen or fixed in 4% formaldehyde. When used, PP2 was dissolved in DMSO and given at a dose of 3 mg/kg in a 0.1-mL volume intraperitoneally 30 minutes before caerulein. Animals pretreated with vehicle were given DMSO only.

### Morphological Examination and Assays

Five μm sections of paraffin-embedded pancreas and lung were stained with hematoxylin/eosin (H&E). For immunohistochemistry of pancreas, 5 μm sections were made from OCT embedded tissue, fixed with 4% paraformaldehyde, and immunostained as described herein. Serum amylase activity was measured colorimetrically using the Phadebas assay (Pharmacia Diagnostics, Portage, Mich.). Edema was measured by subtracting the dry weight from the wet weight and presented as a percentage of wet weight.

### Analysis of Data

The results reported represent the mean±SEM of values obtained from three or more experiments and compared using Student's t test when the data consisted of only two groups or ANOVA when comparing three or more groups. If ANOVA indicated a significant difference, the data were analyzed by using Tukey's method as a post-hoc test for the difference between groups. A p value of 0.05 was considered significant.

### The Src Family Member, Yes, Binds Cortactin and Regulates its Phosphorylation and Localization in Pancreatic Acini

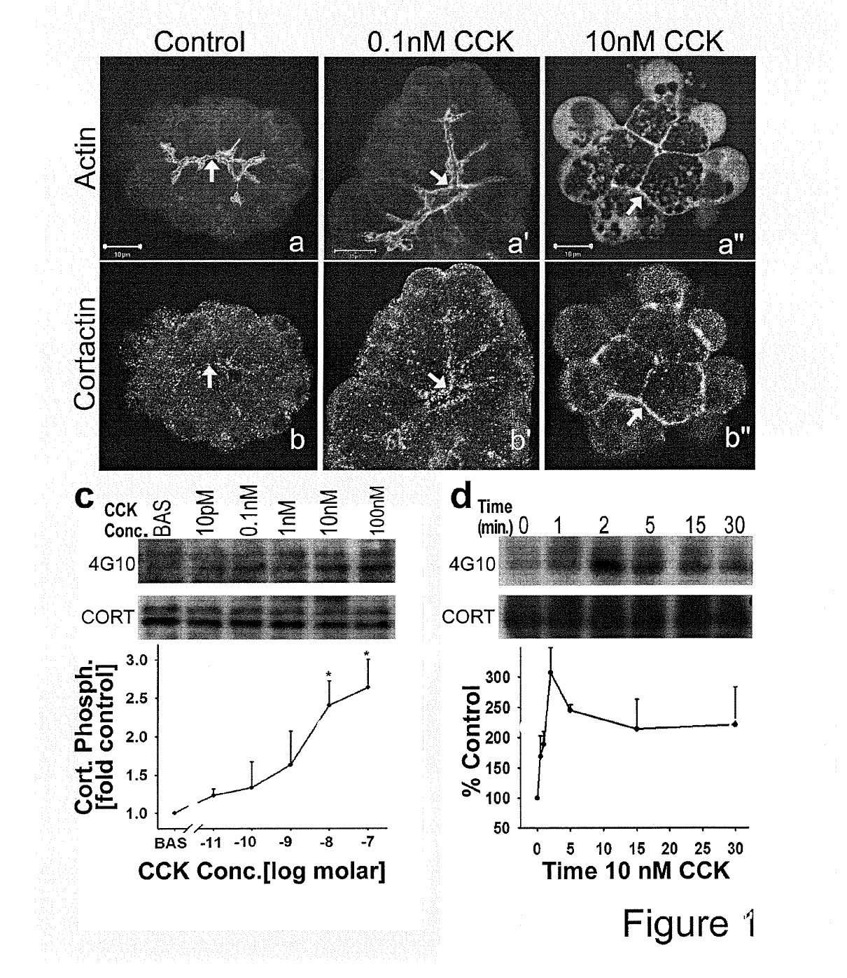

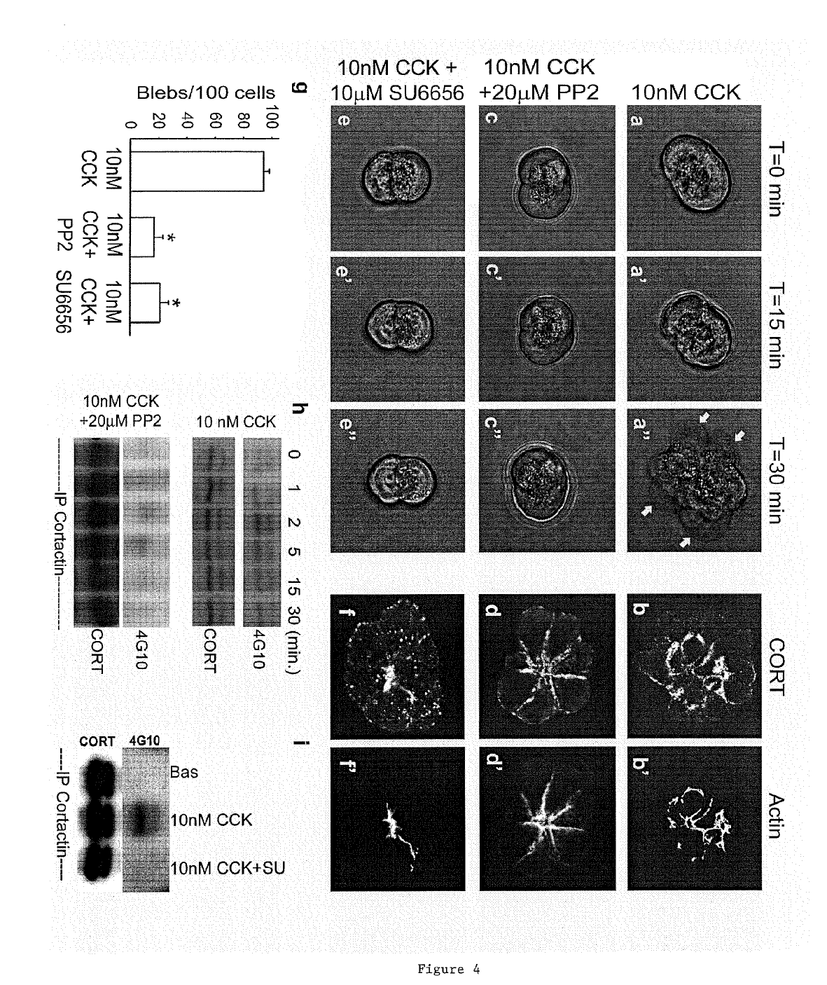

Pancreatic acini relocate their actin basally and form basal blebs in response to supraphysiologic stimulation (10 nM) with CCK (FIG. 1a-a″). Cortactin relocates basally with the actin (FIG. 1b-b″). This basal relocation is associated with cortactin tyrosine phosphorylation, which occurs at supraphysiologic concentrations (FIG. 1c; 2.5±0.2 fold at 10 nM CCK, from three separate experiments). The phosphorylation peaked at two minutes and was sustained over 30 minutes (FIG. 1d; 2.4±0.6 fold at 30 minutes). To determine if Src is mediating this phosphorylation, the acini were pre-incubated with src inhibitors (PP2 at 20 μM or SU6656 at 10 μM), and cortactin phosphorylation was measured. Both PP2 and SU6656 prevented cortactin phosphorylation (FIGS. 4g and 4i). This inhibition was sustained over 30 minutes.

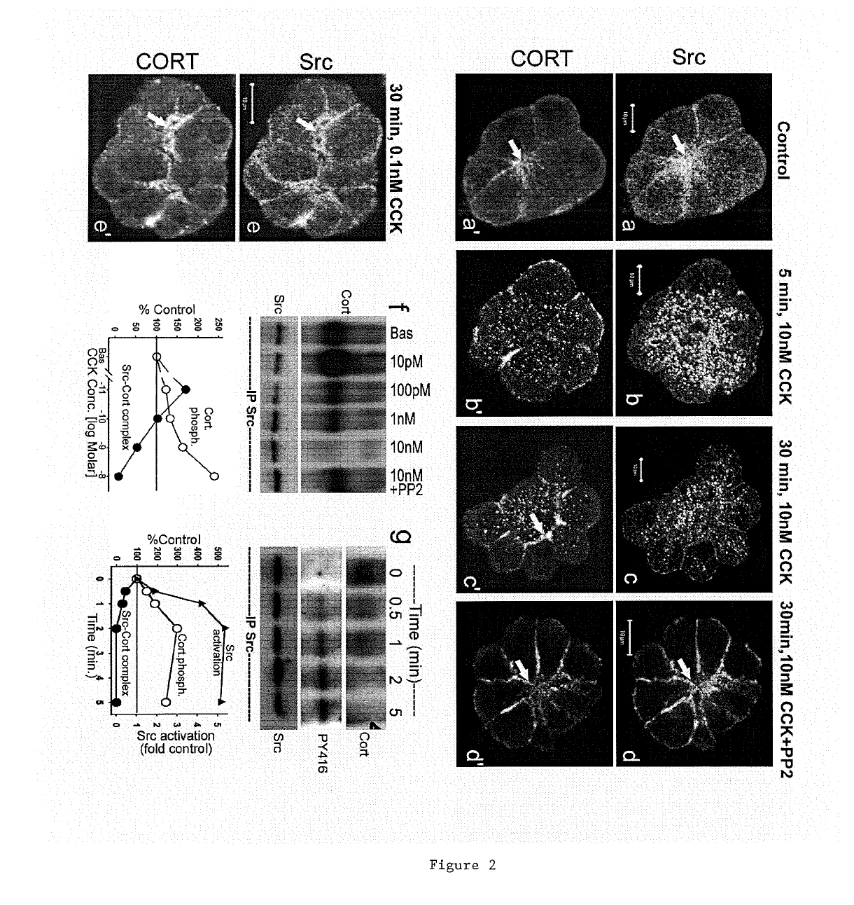

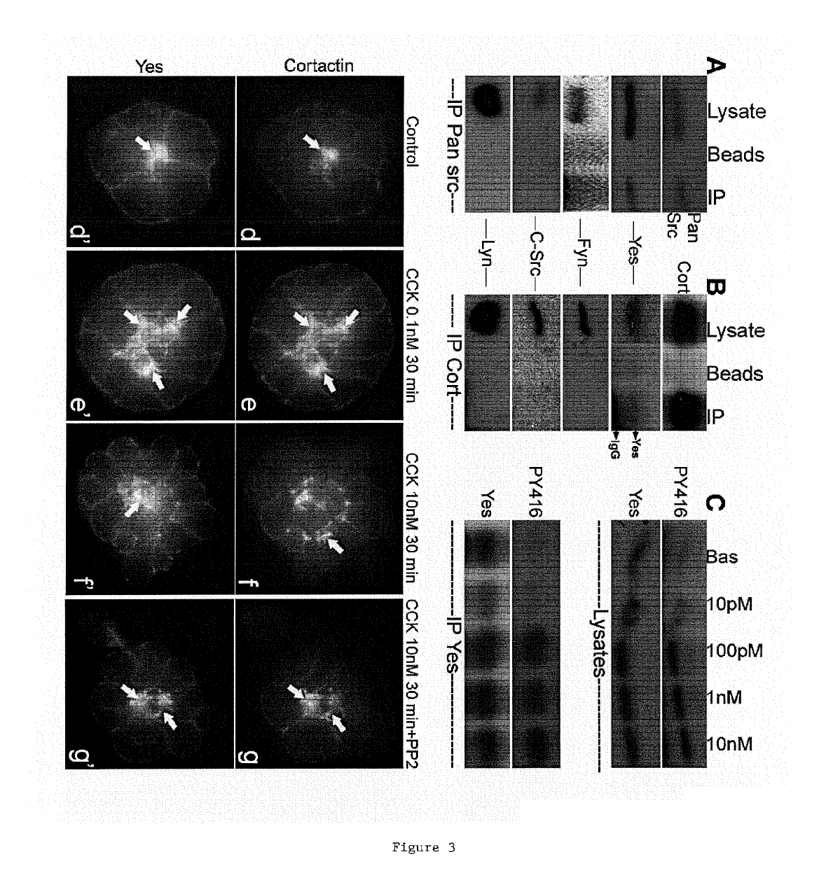

Results using the inhibitors indicated that src can be mediating cortactin phosphorylation. Src was immunoprecipitated, and the association of cortactin with Src was examined. Src and cortactin were found to exist as a complex in the resting state (FIGS. 2f and 2g). Suprastimulation with 10 nM CCK resulted in activation of src (FIG. 2g), phosphorylation of cortactin, and complete dissociation of the complex within two minutes (FIG. 2f). The Src-cortactin complex became progressively weaker with increasing supraphysiologic concentrations of CCK, resulting in complete dissociation of the complex at 10 nM. This dissociation was prevented by PP2 (FIG. 2f last lane). Since large amounts of cortactin seemed to be associated with relatively modest amounts of Src (FIGS. 2f and 2g), demonstrating the reverse phenomenon (i.e., Src co-immunoprecipitating with cortactin) required over expression of cortactin (see FIG. 3a). Using highly concentrated acinar homogenates for immunoprecipitation of large amounts of cortactin resulted in uncontrolled proteolysis. Thus, the association of src with cortactin on immunoprecipitating endogenous cortactin was not determined.

Morphological findings consistent with the dissociation of the src-cortactin complex with suprastimulation and the prevention of the dissociation with Src inhibition were also observed (FIG. 2a-e′). While cortactin rapidly relocated basally within five minutes (FIG. 2b′) and continued to increase on the basal surface (see 30 minutes; FIG. 2c′), Src (stained with the pan Src antibody) remained cytoplasmic and membrane bound without concentrating on the basal surface (FIG. 2b, 2c). PP2 prevented this basal relocation of cortactin (FIG. 2d′) keeping it predominantly localized to the apical surface where it co-localized with src (FIG. 2d), much like in physiological stimulation (FIGS. 2e, e′).

To identify the src family member that associates with cortactin, immunoprecipitates were probed with antibodies to four src family members (Yes, Fyn, c-Src, and Lyn) identified in pancreatic acini (Lynch et al., Pflugers Arch., 447(4):445-51 (2004) and Pace et al., Biochim. Biophys. Acta., 1763(4):356-65 (2006)). The pan Src antibody (SC-18) identifies the C-terminal domain of Src that is common to all family members. Antibodies to the different src family members were made to the N-terminal domain, which is unique to each family member. While acinar cell lysates contained all four family members, the only src family member consistently pulled down with the pan Src antibody was Yes (FIG. 3a). Similarly, immunoprecipitates of cortactin prepared from acinar cells expressing WT cortactin only pulled down Yes (FIG. 3b). Since the putative cortactin binding site of src is included in the epitope of the antibodies made to the family members, co-immunoprecipitation was not possible with these antibodies.

To compare the dose response of Src and Yes activation, lysates prepared from acini stimulated with different concentrations of CCK were blotted for active Src (PY416) and reprobed for Yes. The band for active Src was identical to that of Yes (FIG. 2c, upper panel). Supporting this, when Yes was immunoprecipitated from the same lysates and blotted for its activation (FIG. 3c, lower panel), the pattern and extent of Yes activation was identical to that seen in whole cell lysates, with both occurring at 100 pM, remaining sustained over the suprastimulation range, and being 2.7 fold at 10 nM.

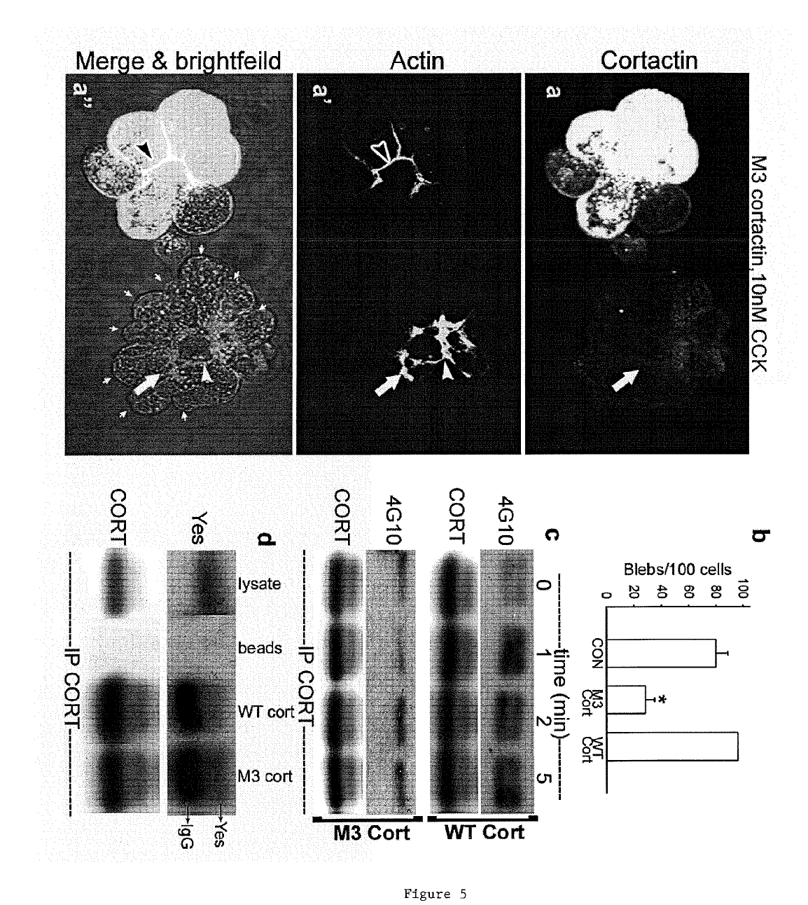

To examine Yes localization, simultaneous staining for Yes and cortactin was performed (FIG. 3d-g). Yes exhibited more membranous localization like that of cortactin compared to the pan src antibody. Moreover, Yes staining remained apical with physiologic CCK (0.1 nM, FIG. 3e′) and supraphysiologic CCK (FIG. 3f), while cortactin relocated basally with supraphysiologic CCK (10 nM, FIG. 3f) similar to the observed src-cortactin dissociation at supraphysiologic concentrations (FIG. 2). PP2, which prevented cortactin phosphorylation by Src, prevented this relocation, thereby recapitulating the morphological and biochemical behavior of Src and cortactin observed in FIG. 2. M3 cortactin, which is refractory to phosphorylation by Src, remained associated with Yes despite supraphysiologic stimulation (FIG. 5c). It is hypothesized that Yes and cortactin exist as a complex, and tyrosine phosphorylation of cortactin by Yes under supraphysiologic concentrations of CCK results in its dissociation from Yes and basal localization, with consequent basal actin relocation and blebbing.

### Phosphorylation of Cortactin by Yes Results in Basal Actin Localization and Acinar Cell Blebbing

Since cortactin is a major actin regulatory protein and relocates to the basal surface along with actin when phosphorylated by Yes, the hypothesis that preventing cortactin phosphorylation can prevent basal localization of actin and consequent bleb formation was examined. Two approaches were used: (1) the use of src inhibitors and (2) the use of a dominant negative cortactin (M3 cortactin), which is refractory to activation by src.

Both PP2 (20 μM) and SU6656 (10 μM), which completely prevented cortactin phosphorylation (FIG. 4h, 4i), prevented the basal relocation of cortactin (compare FIG. 4d, f to FIG. 4b) and actin (compare FIG. 4d′ and f′ to Figure b′). This was associated with a significant reduction in blebbing from 94.2±4.1% to 16.9±6.0% and 21.0±6.0% with PP2 and SU6656, respectively (FIG. 4g). FIGS. 4a-a″ reveal this phenomenon as seen using phase imaging after 0, 15 minutes, and 30 minutes in a cell suprastimulated with 10 nM CCK. FIGS. 4c-c″ and FIGS. 4e-e″ contain corresponding images of PP2 and SU6656 preventing blebbing.

To determine if the prevention of blebbing by src inhibitors was due to cortactin not being phosphorylated, WT cortactin and M3 cortactin, where the tyrosines at positions 384, 429, and 445 were mutated to phenylalanine, were over expressed. The acinus over-expressing M3 cortactin (FIG. 5a-a″, left side) kept its actin apical (outlined arrowhead) as compared to the untransfected acinus (right side) in which the actin translocated basally (large Arrow), with consequent formation of large blebs (small arrows). This prevention of suprastimulation induced blebbing in the M3 cortactin transfected cells was shown graphically in FIG. 5b (28.1±6.6% in M3 cortactin vs. 79.3±8.8% in untransfected cells). WT cortactin did not prevent blebbing (96.0±0.1%). While WT cortactin was promptly phosphorylated by suprastimulation (FIG. 5c upper panel), M3 cortactin remained refractory to tyrosine phosphorylation by CCK (FIG. 5c, lower panel). This was not due to lack of src activation, since src activation in response to CCK was observed in the M3 transfected cells.

While both WT and M3 cortactin bind Yes under unstimulated conditions, WT cortactin was phosphorylated within one minute of CCK stimulation and dissociated from Yes, while M3 cortactin continued to be associated with Yes (FIGS. 3b and 5d). To determine if cortactin phosphorylation is responsible for the basal relocation of actin and consequent cell blebbing, suprastimulated cells over expressing WT cortactin and M3 cortactin were stained with Rhodamine phalloidin. It was hypothesized that src inhibition would prevent corresponding changes in vivo and reduce the severity of pancreatitis.

Inhibition of Src Prevents Cortactin Phosphorylation and Actin Redistribution During Pancreatitis, with Consequent Decrease in its Severity.

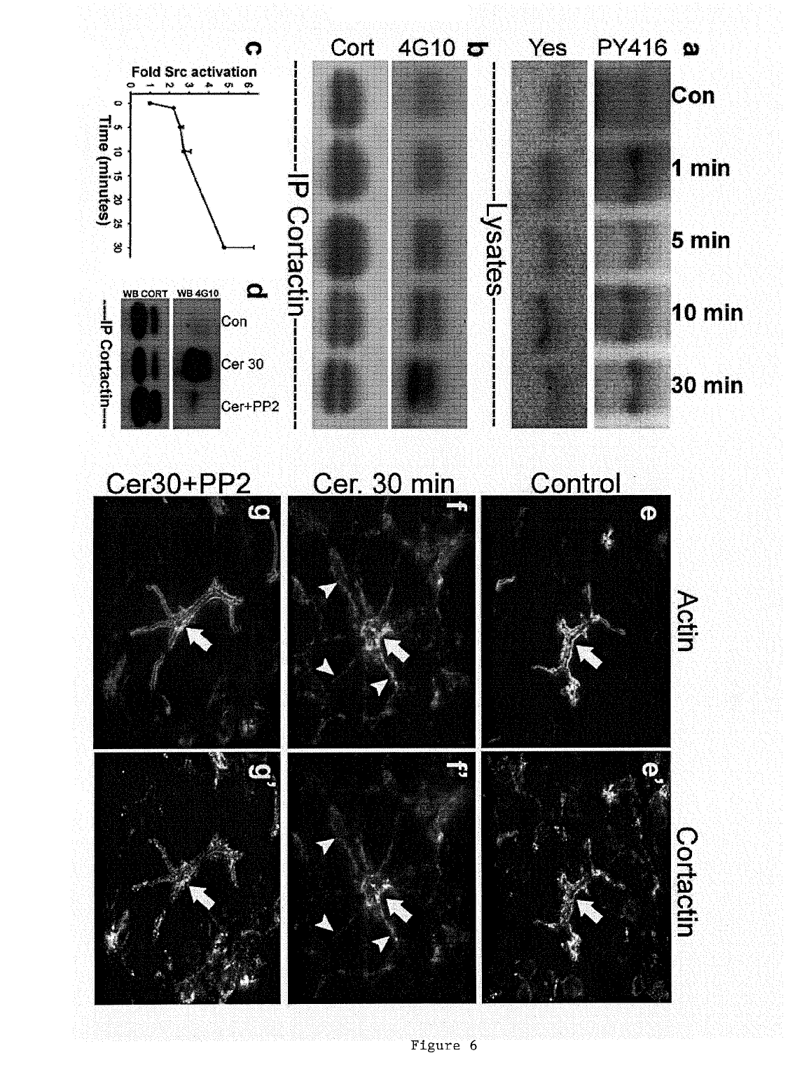

To determine if src inhibition reduces the severity of pancreatitis, the time course of src activation and cortactin phosphorylation in vivo in response to a 20 μg/kg intraperitoneal injection of the CCK analogue caerulein (CER) was examined. In particular, the time course of Yes activation in the lysates of pancreata harvested at various times from different animals was determined (FIG. 6a). There was a 2.2 fold activation of src within one minute, and this activation was sustained over 30 minutes (5.0 fold). When cortactin was immunoprecipitated from these lysates and blotted for phosphotyrosine using an 4G10 antibody, there was a significant increase in its tyrosine phosphorylation noted at five minutes, and this was sustained over 30 minutes (FIG. 6c). Given that the activation of src and tyrosine phosphorylation of cortactin occurred so early, the src inhibitor was administered prophylactically. Preliminary data revealed that 3 mg/kg of PP2 given intraperitoneally was adequate to inhibit cortactin phosphorylation (FIG. 6d).

To determine if PP2 would prevent the changes corresponding to actin and cortactin regarding the location noted in vitro when given to live animals, cryosections of the pancreas were immunostained for actin and cortactin. Normal pancreas exhibited rich apical actin and cortactin staining (FIG. 6 e and e′, respectively). They relocated basolaterally within 30 minutes of a caerulein injection (FIGS. 6f and f′, respectively). PP2, when administered prophylactically, prevented this basolateral relocation (FIGS. 6g and g′).

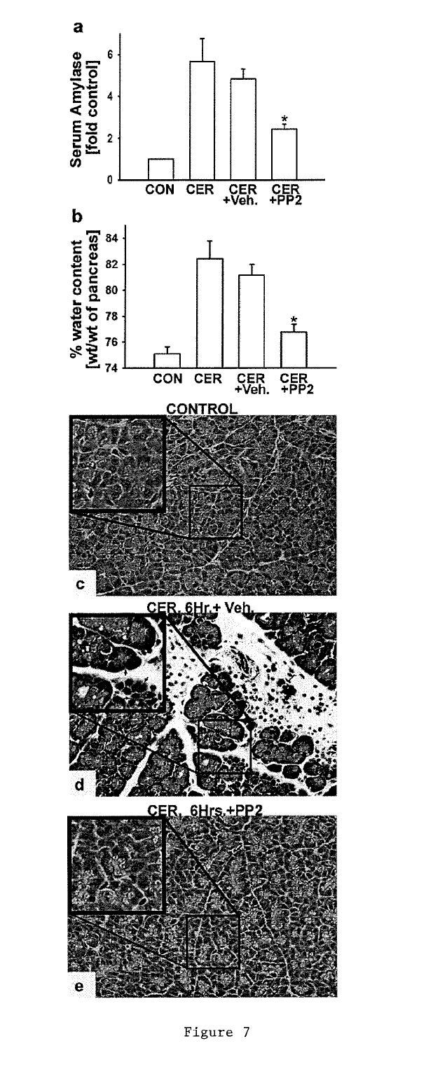

Since PP2 was effective in preventing cortactin phosphorylation and cytoskeletal changes during pancreatitis, its efficacy in reducing the extent of acinar cell injury during pancreatitis was examined. The effect of PP2 on serum amylase after 6 hours of pancreatitis was determined (FIG. 7a). Animals with pancreatitis (CER) had a 5.7±1.0 fold increase in serum amylase over controls (CON). This was not significantly influenced by prophylactic administration of the vehicle (CER+Veh, 4.8±0.5 fold). PP2 prevented this increase in serum amylase significantly (CER+PP2, 2.4±0.2 fold, p value <0.02). Similarly, PP2 caused a significant reduction in pancreatic edema as measured by water content (FIG. 7b) from 81.1±0.8% (CER+Veh) to 76.7±0.6% CER+PP2 (p<0.05). Control pancreatic water content was 75.1±0.5%.

PP2 also improved the morphology of the pancreas. While there was vacuolation, rounded fragmentation of the basal cytoplasm of acinar cells akin to blebbing (arrows in inset of FIG. 7d), apoptosis (see inset FIG. 7d), interstitial edema, and inflammatory cells in the pancreas of animals with pancreatitis or those given vehicle (CER6 Hr.+Veh., FIG. 7d), PP2 dramatically prevented these phenomena (FIG. 7e).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having pancreatitis, wherein said method comprises administering a tyrosine kinase inhibitor having the ability to inhibit phosphorylation of cortactin to said mammal under conditions wherein the severity of a symptom of said pancreatitis is reduced.

2. The method of claim 1, wherein said mammal is a human.

3. The method of claim 1, wherein said pancreatitis is acute pancreatitis.

4. The method of claim 1, wherein said pancreatitis is chronic pancreatitis.

5. The method of claim 1, wherein said tyrosine kinase inhibitor is selected from the group consisting of SKI-606, BMS354825, AZD0530, AP23464, CGP76030, AMN107, PP1, PP2, SU6656, and Imatinib mesylate.

6. The method of claim 1, wherein said method comprises administering a composition comprising two or more tyrosine kinase inhibitors having the ability to inhibit phosphorylation of cortactin.

7. The method of claim 1, wherein the severity of said symptom is reduced by at least 25 percent.

8. The method of claim 1, wherein the severity of said symptom is reduced by at least 50 percent.

9. The method of claim 1, wherein the severity of said symptom is reduced by at least 75 percent.

10. The method of claim 1, wherein said method comprises identifying said mammal as having said pancreatitis before said administering step.

11. The method of claim 1, wherein said method comprises monitoring said mammal for said reduction in the severity of said symptom after said administering step.
